(19) United States
(12) Patent Application Publication
(10) Pub. No.: US 2006/0099703 A1
(43) Pub. Date: May 11, 2006

(54) Device for quantitative analysis of biological materials

(75) Inventors: In-Hwan Choi, Yoning-si (JP); Jin-Woo Lee, Suwon-si (JP); Seung-Joo Xang, Seoul (KR)

Correspondence Address: MACPHERSON KWOK CHEN & HEID LLP, 1762 TECHNOLOGY DRIVE, SUITE 226, SAN JOSE, CA, 95110, US

(73) Assignee: ALL MEDICUS CO., LTD, Kyunggi-do (KR)

(21) Appl. No.: 10/520,555

(22) PCT Filed: Jun. 30, 2003
(86) PCT No.: PCT/KR03/01280

§ 371 Date: Dec. 1, 2005

(30) Foreign Application Priority Data
Jul. 5, 2002 (KR) 10-2002-0039072

(51) Int. Cl. C12M 1/34

(52) U.S. Cl. 435/287.1

(57) ABSTRACT

Disclosed is a device for quantitatively analyzing material of a living creature. The device includes an analyzing unit using photometric method, and an analyzing unit using electrochemical method. The device has a socket for mounting a photometric test strip and a socket for mounting an electrochemical test strip, separately or in a body. Also, the test strip may have a recognition electrode formed on a specific position of the test strip. The position of the recognition electrode is determined by analyzing method and target material. When used together with a test strip having the recognition electrode, the device has a terminal for identifying the position of the recognition electrode.

### TECHNICAL FIELD

This invention relates to a device for quantitatively analyzing biochemical components (analytes), particularly to a device, which is used together with a test strip, for quantitatively analyzing biochemical components in aqueous fluids, particularly whole blood, according to the reaction result between the reagent attached to the test strip and the biochemical components. This invention is useful in quantitatively analyzing biochemical components such as glucose, cholesterol, lactate, and so forth, selectively.

### BACKGROUND ART

Recently many methods have been proposed in medical field to analyze biochemical components including blood. Among those, biosensors utilizing enzyme analysis are used most widely in hospital and clinical laboratories because they are easy to apply, superior in measurement sensitivity, and allow rapid acquisition of test result. The enzyme analysis applied in biosensor can be broadly divided into a photometric method and an electrochemical method. The photometric method is described in U.S. Pat. No. 4,935,346, and the electrochemical method is described in U.S. Pat. No. 5,997,817.

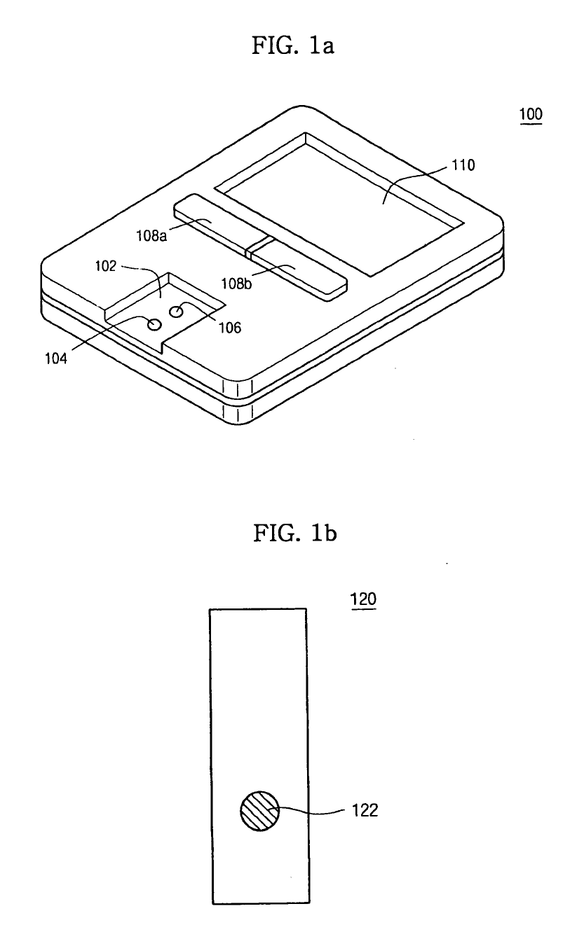

FIG. 1a is a schematic view of a conventional device using the photometric method, and FIG. 1b is a schematic view of a test strip used with the device of FIG. 1a. In the photometric device 100, the light-emitting element 104, such as a light-emitting diode (LED) and the light-detecting element 106 are installed below the bottom surface of the mounting part 102 on which test strip 120 is mounted. A reagent reacting to biochemical components, especially material which is a target for analysis (target material) is fixed in the reaction region 122 of the test strip 120. When a user turns on the device 100 by handling the buttons 108a and 108b, the device 100 senses whether a test strip is installed or not. Moreover, if the device 100 has a function of analyzing various biochemical components, the user provides information about target material to the device 100 by handling of buttons 108a, 108b. As the reaction between the reagent of the reaction region 122 and biochemical components progresses, the color of the reaction region 122 varies gradually. The color variation of the reaction region 122 is measured as the degree that light emitted from the light-emitting element 104 is detected at the light-detecting element 106 after being reflected from the reaction region 122. The measurement result is analyzed by a built-in analysis algorithm (not shown) and displayed on a monitor, such as a liquid crystal display (hereinafter referred to as “LCD”).

Those photometric biosensors have been developed over various biochemical components because those can be easily embodied. However, the measuring time of the photometric biosensors is longer than that of the electrochemical biosensors. Also, since the photometric biosensors have generally measurement errors caused by the turbidity of the biochemical components, it is sometimes very difficult to analyze significant biochemical components using the photometric biosensors. Moreover, it is difficult to discriminate whether a test strip is installed in a photometric biosensor. Therefore, the electrochemical biosensors have been widely used recently. In an electrochemical biosensor, an electrochemical system is made on a nonconductive substrate by screen printing or physical vapor deposition, and a reagent is fixed at a specific region (reaction region) on the electrochemical system. In measuring biochemical components, a predetermined level of voltage is applied to the electrochemical system, and a sample including the biochemical components is introduced at the reaction region.

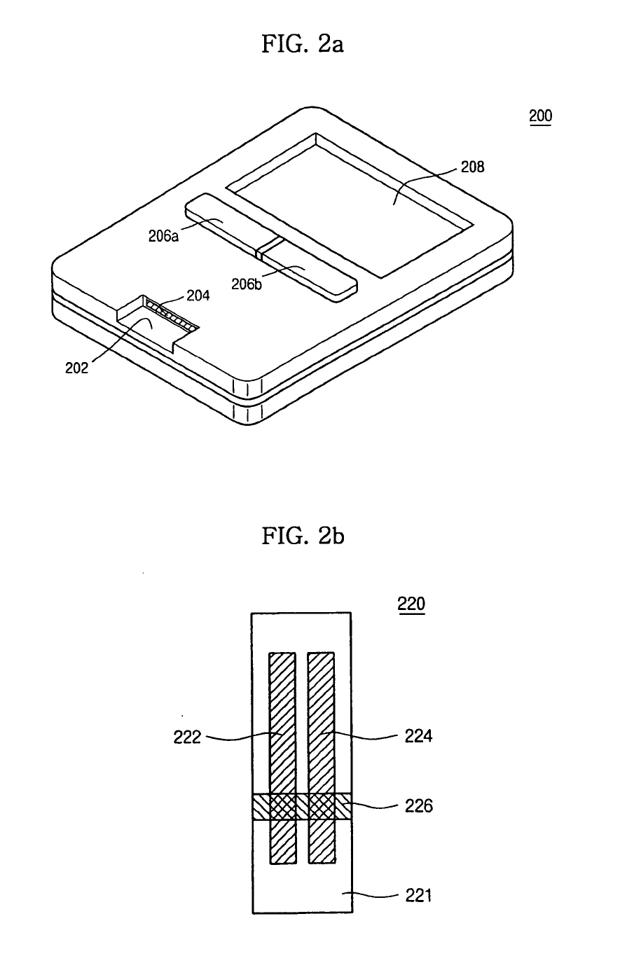

FIG. 2a is a schematic view of a conventional device using the electrochemical method, and FIG. 2b is a schematic view of the electrochemical test strip used with the device of FIG. 2a. The device 200 using the electrochemical method has a socket (not shown) electrically connected with the electrodes of the test strip 220. The electrodes 222 and 224 of the test strip 220 are connected with the terminals formed in the socket of the device 200, when the test strip 220 is inserted into the device 200 through the insertion hole 204. The mounting part 202 supports the test strip 220, which is physically weak. After a user turns on the device 200 by handling the buttons 206a and 206b, the device 200 receives information about target material to be analyzed trough the user's additional handling of the buttons 206a and 206b. As shown in FIG. 2b, the test strip 220 is formed by depositing the reference electrode 222 and the working electrode 224 on the insulating substrate 221, and fixing reagent 226 across the reference electrode 222 and the working electrode 224. The oxidation-reduction (redox) reaction between the target material and the reagent 226 occurs. Generally, a capillary is formed on the reagent 226 by depositing another insulating substrate (not shown) on the insulating substrate 221 in order that the target material permeates appropriately into the entire reagent 226. The reaction on the test strip 220 between the reagent 226 and the target material, by means of an electrochemical mechanism, allows current to flow in the working electrode 224. Since the density of the target material determines the intensity of the flowing current, an analysis algorithm calculates the density of the target material by measuring the value of the flowing current. The density is displayed on the LCD 208.

The electrochemical biosensors are easy to use, and superior in measurement accuracy. Also, the measuring time of the electrochemical biosensors is generally shorter than that of the photometric biosensors. Moreover, the electrochemical biosensors can generally identify easily whether the test strip is mounted on the device. However, the electrochemical biosensors have a defect that those can be applied only a few biochemical components, because those are not developed over various biochemical components compared with the photometric biosensors.

As mentioned above, a biosensor utilizing enzyme analysis comprises a test strip and a device (meter). In the test strip, a reagent reacting with target material is fixed. Since the device has a processing unit according to an analysis method, it can analyze the reaction result on the test strip with a photometrical or an electrochemical method, and display the result in a form that a user can utilize. Because the reagent, which is fixed to the test strip, is decided according to target material, the test strip is exclusively used for the target material. However, a device can be used for various biochemical components if it has a processing unit that can handle various biochemical components and if it can selectively operate the processing unit according to target material.

In case of analyzing various biochemical components using one device, a button is generally used to inform the device of information about target material. However, the button is inconvenient to use. For example, in case of glucose measuring sensor, considering that major users are the elderly, they feel even simple manipulation to be difficult and inconvenient. Accordingly, a code chip is developed to overcome this problem. The code chip has an advantage that it can inform the device of much information including target material. However, considering the cost of a conventional test strip is so low, the manufacturing cost of the code chip is really very expensive in the market.

### DISCLOSURE OF THE INVENTION

An object of the present invention, which is proposed to solve these problems, is to provide biochemical components measuring device that can realize electrochemical method and photometric method in one system for analyzing biochemical components.

Another object of the present invention is to provide biochemical components measuring device that is easy to use and inexpensive.

To achieve the above-mentioned objects, the present invention provides a biochemical components measuring device that has both a processing unit for analyzing biochemical components with a photometrical method and a processing unit for analyzing biochemical components with an electrochemical method. The device may has separately a socket for mounting a photometric test strip and a socket for mounting an electrochemical test strip, or has a socket for mounting both types of test strip.

The photometric test strip according to the present invention may have a recognition electrode formed at a specific position determined by target material and analysis method, on the test strip. In this case, in order to identify the position of the recognition electrode, plural terminals for the recognition electrode are installed in the socket for photometric method. The number of the variable positions of the recognition electrode determines the number of the terminals for the recognition electrode. A built-in switch for discerning whether the photometric test strip in which a recognition electrode is not formed, is mounted or not may be installed in the socket for photometric method of the biochemical components measuring device. Also, the electrochemical test strip according to the present invention may have a recognition electrode formed at the specific position determined by target material and analysis method. In this case, in order to identify the position of the recognition electrode, plural terminals for the recognition electrode are installed in the socket for electrochemical method.

If photometric method and electrochemical method are embodied in a device according the present invention, a user can use all the advantages of the two methods. In other words, for some materials to which electrochemical method can be applicable, a user can perform the analysis of high accuracy by using the electrochemical method, and perform analysis for other various materials by using the photometric method.

Since the recognition electrode of the test strip indicates information about target material and analysis method, the device according to the present invention can identify target material and analysis method by checking the position of the recognition electrode. After identifying whether the mounted test strip is for photometric method or electrochemical method, the device carries out the corresponding processing routine. Therefore, the device does not need to manipulate a button to provide information about target material and analysis method to the device. Consequently, the device according to the invention is very convenient to use.

In addition, the recognition electrode according to the present invention is merely an electrode formed at a specific position on the test strip. Therefore, compared with the conventional manufacturing process, there is substantially no need of an additional process in manufacturing a test strip. Moreover, it is very advantageous that two measuring methods can be used not through two separate systems, but through only one system.

### BRIEF DESCRIPTION OF THE INVENTION

FIG. 1 shows a conventional device and a test strip using the photometric method.

FIG. 2 shows a conventional device and a test strip using the electrochemical method.

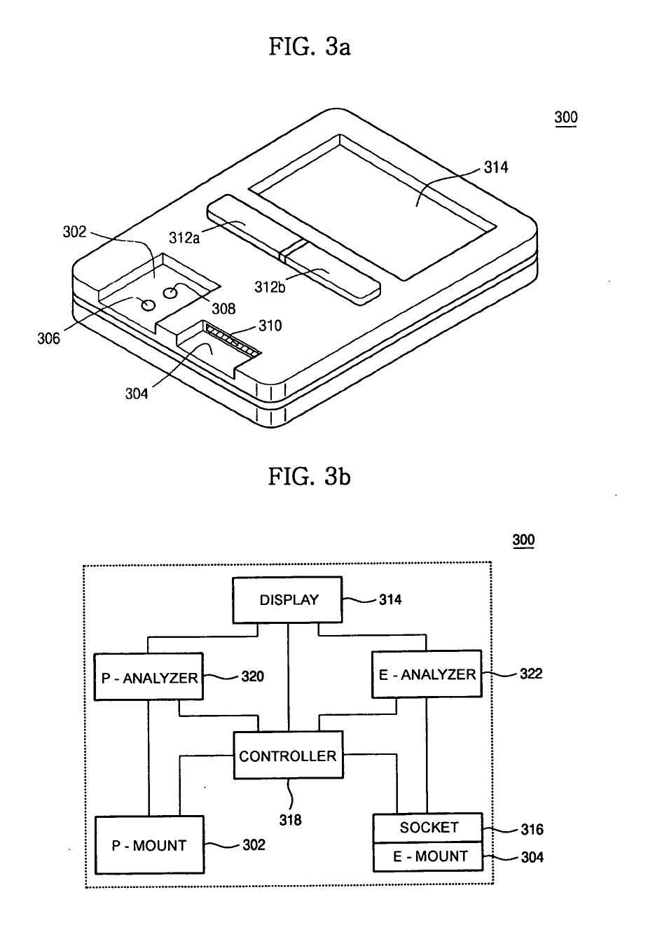

FIG. 3 is a schematic view and a block diagram of the device according to an embodiment of the invention.

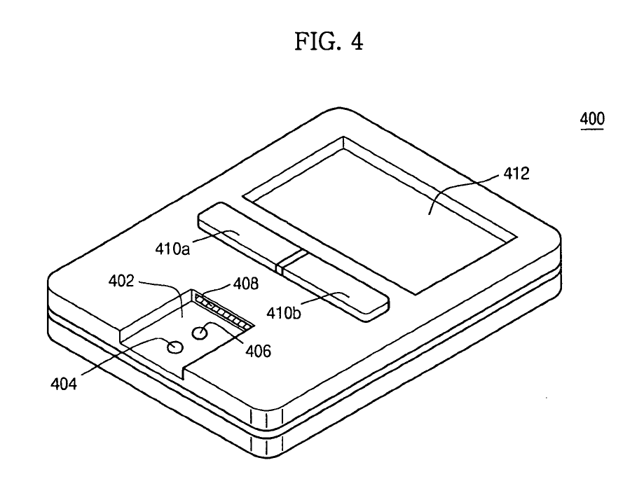

FIG. 4 is a schematic view of the device according to another embodiment of the invention.

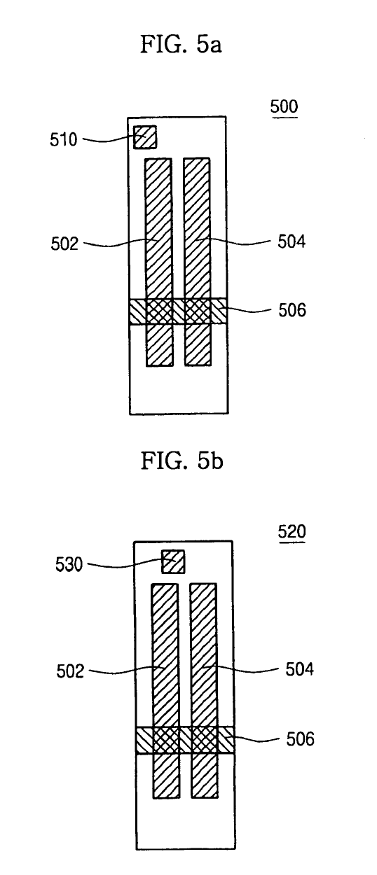

FIG. 5 shows an exemplary test strip according to the invention.

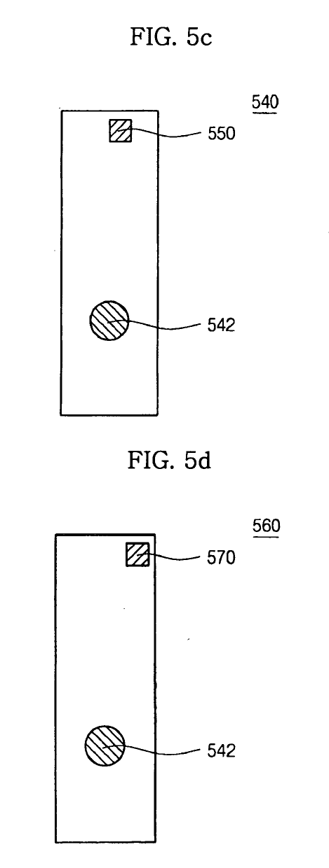

FIG. 6 shows an exemplary socket of the device according to the invention.

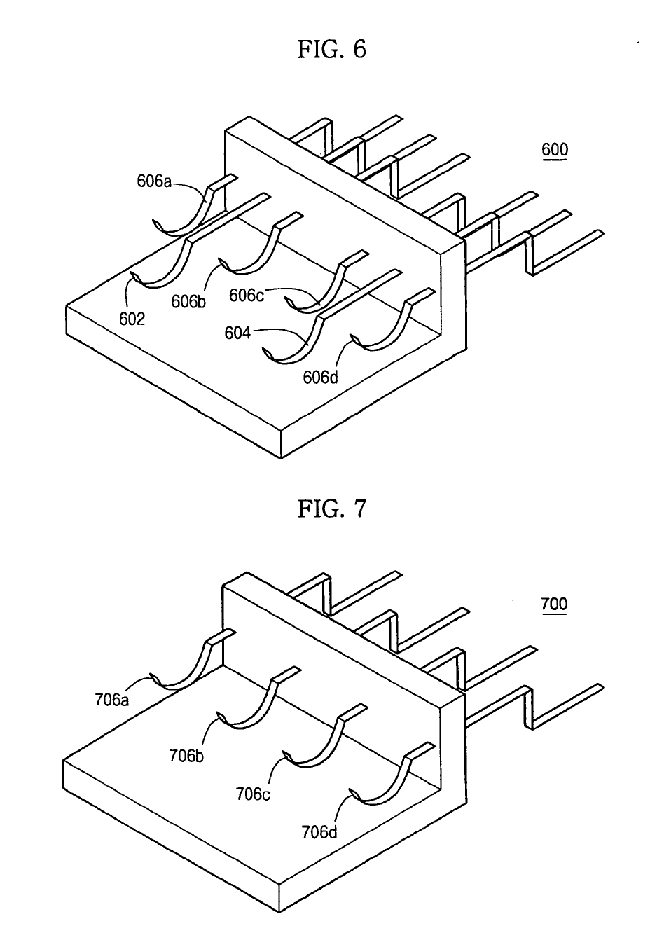

FIG. 7 shows a second exemplary socket of the device according to the invention.

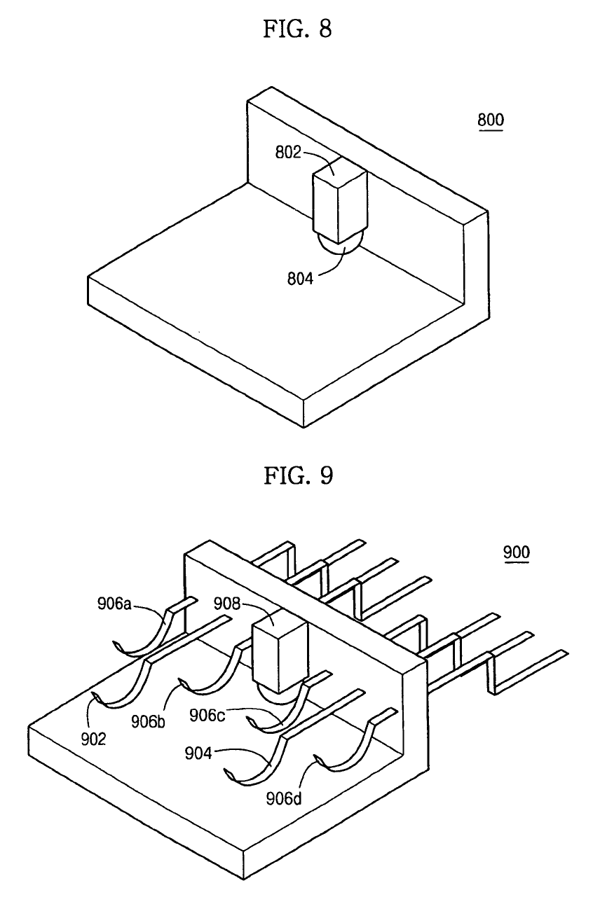

FIG. 8 shows a third exemplary socket of the device according to the invention.

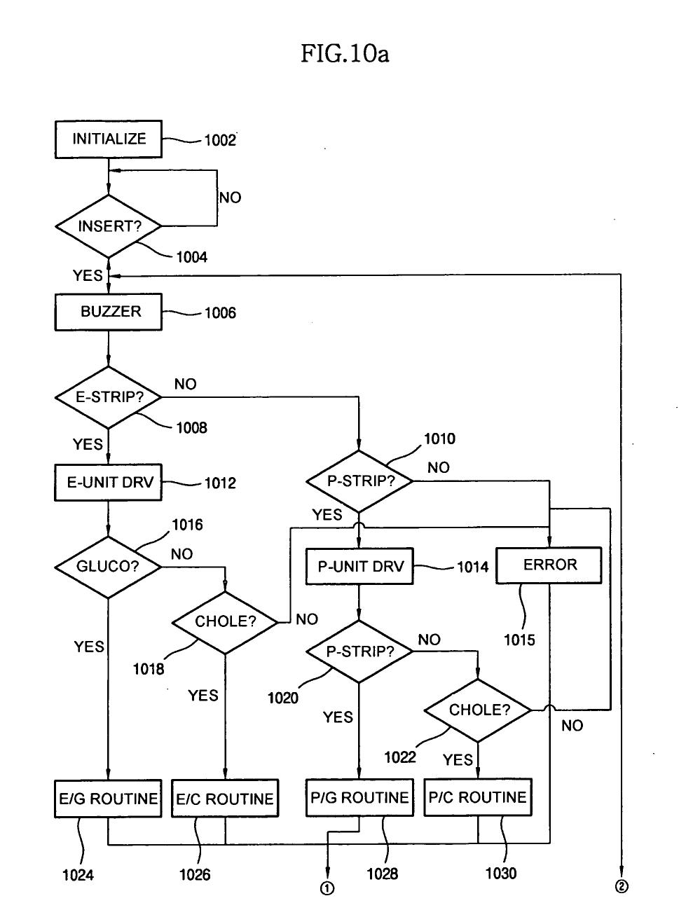

FIG. 9 shows a fourth exemplary socket of the device according to the invention.

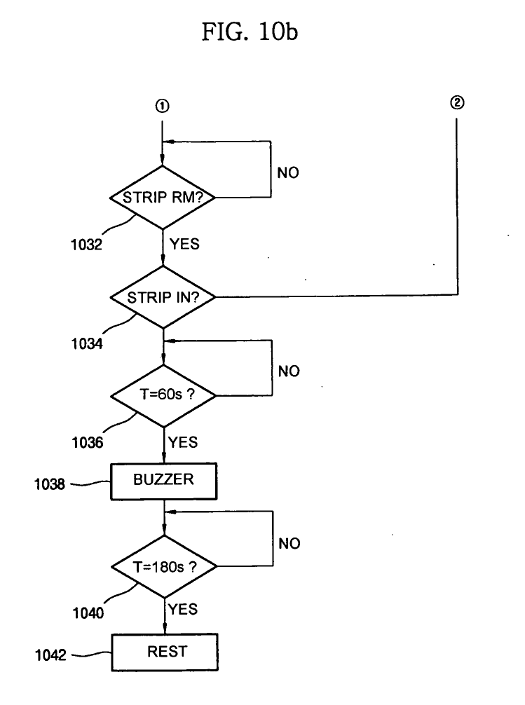

FIG. 10 shows a flow chart explaining the control method of the device according to the invention.

### BEST MODE FOR CARRYING OUT THE INVENTION

Hereinafter, the present invention is described specifically with reference to the embodiments illustrated in the drawings. But the embodiments below are to describe the invention in detail, not to limit the range of the invention.

FIG. 3a is a schematic view of the device according to an embodiment of the invention. As shown in FIG. 3a, the device 300 has both a mounting part 302 for mounting a photometric test strip and a mounting part 304 for mounting an electrochemical test strip. As illustrated previously in connection with FIG. 1, the light-emitting element 306 and the light-detecting element 308 are installed in the mounting part 302. The electrochemical test strip installed in the mounting part 304 is electrically connected with a built-in socket (not shown) through the insertion hole 310. Through the manipulation of the buttons 312a and 312b, the device 300 is turned on or off and one of various function modes is selected. The analysis result from the device 300 is generally displayed on LCD 314.

The biochemical components that can be analyzed with an electrochemical method can be analyzed by mounting an electrochemical test strip on the mounting part 304. On the other hand, the biochemical components that a photometric method should be applied to, can be analyzed by mounting a photometric test strip on the mounting part 302. If the device 300 is used, a user can selectively use a photometric method and an electrochemical method in merely one device. Therefore, it is possible to analyze various biochemical components and to analyze some biochemical components more accurately.

FIG. 3b is a block diagram of the device 300 shown in FIG. 3a. As sown in FIG. 3b, the device 300 has both the mounting part 302 for mounting a photometric test strip and the mounting part 304 for mounting an electrochemical test strip. The light-emitting element 306 and the light-detecting element 308 are installed in the mounting part 302 as shown in FIG. 3a. Also, the socket 316 that is electrically connected with the electrodes of an electrochemical test strip adjoins the mounting part 304. The controller 318 controls all the operations of the device 300. The analyzer 320 analyzes biochemical components according to a predetermined photometric method, by using the results detected from the photometric test strip mounted in the mounting part 302 through the light-emitting element 306 and the light-detecting element 308. And the analyzer 322 analyzes biochemical components according to a predetermined electrochemical method, by using the results detected from the electrochemical test strip mounted in the mounting part 304 through the socket 316.

Referring to FIG. 3b, the analyzer 320 performing a photometric analysis and the analyzer 322 performing an electrochemical analysis appear to be physically separated. However, this is nothing but a conceptual illustration for the description. Generally, the measuring device stores both a photometric analyzing algorithm and an electrochemical analyzing algorithm in one memory unit (not shown). And when a photometric test strip is mounted on the mounting part 302, the photometric analyzing algorithm is read from the memory unit and performed. However, when an electrochemical test strip is mounted on the mounting part 304, the electrochemical analyzing algorithm is read from the memory unit and performed. The controller 318 controls the execution of the analysis algorithms.

FIG. 4 is a schematic view of the device according to another embodiment of the invention. As shown in FIG. 4, the device 400 has the mounting part 402 for mounting both a photometric test strip and an electrochemical test strip selectively. The others are the same as that of the device 300 shown in FIG. 3. Since the device 300 shown in FIG. 3 has two mounting parts, a user should discriminate mounting parts according to analysis method. However, the device 400 shown in FIG. 4 has just one mounting part. Thus, because there is no need to discriminate mounting parts according to analysis method, the device is easy to use and can be manufactured more compactly.

FIG. 5 is a schematic view of an exemplary test strip according to the invention. FIG. 5a is an electrochemical test strip for measuring glucose, and FIG. 5b is an electrochemical test strip for measuring cholesterol. Also, FIG. 5c is a photometric test strip for measuring glucose, and FIG. 5d is a photometric test strip for measuring cholesterol. The electrochemical test strips 500 and 520 include a reference electrode 502, a working electrode 504, and reagent 506 attached across the reference electrode 502 and the working electrode 504, as previously described referring to FIG. 2b. And the photometric test strips 540 and 560 include a reaction region 542, as previously described referring to FIG. 1b.

The recognition electrodes 510, 530, 550 and 570 are formed at a specific position of the upper part on the test strip 500, 520, 540 and 560 respectively. The recognition electrodes 510, 530, 550 and 570 indicate what material can be analyzed using the test strip, and whether the test strip is for the photometric method or the electrochemical method. Moreover, the recognition electrodes 510, 530, 550 and 570 provide the measuring device about whether a test strip is inserted or not. The recognition electrodes 510, 530, 550 and 570 are discriminated by their positions formed on the test strips 500, 520, 540 and 560, respectively. The position of the recognition electrode 510 indicates that the test strip 500 is an electrochemical test strip for measuring glucose. In the test strip 520, the position of the recognition electrode 530 is formed on a slight right side as compared with the recognition electrode 510. The recognition electrode 530 indicates that the test strip 520 is an electrochemical test strip for measuring cholesterol. Similarly, the position of the recognition electrode 550 indicates that the test strip 540 is a photometric test strip for measuring glucose. As shown in FIG. 5c, the recognition electrode 550 is formed on a slight right side as compared with the recognition electrode 530. The test strip 560 shown in FIG. 5d is a photometric test strip for measuring cholesterol. As shown, the recognition electrode 570 is formed on a slight right side as compared with the recognition electrode 550.

The recognition electrodes of this embodiment are classified into four classes, according to the position. However, it is possible to make the recognition electrodes having a great variety of information by narrowing the width of the recognition electrodes and/or arranging the recognition electrodes two-dimensionally on the test strip. Also, although the test strips shown in FIGS. 5a and 5b correspond to a two-electrode system, the invention is applicable to a three-electrode system similarly.

FIG. 6 shows an exemplary socket of the measuring device according to the invention. In the socket part 600 shown in FIG. 6, the terminal 602 is electrically connected with the reference electrode 502 of the electrochemical test strips 500, 520 shown in FIGS. 5a and 5b. The terminal 604 is electrically connected with the working electrode 504. And the terminals 606a-606d is electrically connected with the recognition electrodes 510, 530, 550, and 570 respectively. For example, when the test strip 500 shown in FIG. 5a is mounted on the measuring device, the recognition electrode 510 is electrically connected with the terminal 606a of the socket 600, and the device identifies that the electrochemical test strip for measuring glucose is inserted. If the test strip 560 shown in FIG. 5d is mounted, the recognition electrode 570 is electrically connected with the terminal 606d of the socket 600, and the device identifies that the photometric test strip for measuring cholesterol is inserted. In other words, the measuring device can identify analysis method and target material of the test strip through the position of the terminal that is electrically connected with the recognition electrode of the inserted test strip. The conventional photometric test strip does not have the reference electrode 222 and the working electrode 224 differently from the electrochemical test strip shown in FIG. 2b. Only the reagent is fixed on the reaction region 122 as shown in FIG. 1b. Therefore, in case that a photometric test strip is mounted, the device has a difficulty in identifying the insertion of a test strip. However, if a photometric test strip has a recognition electrode 550 and 570 as shown in FIG. 5c and FIG. 5d, the device can easily identify the insertion of the photometric test strip 540 and 560 by checking electrical connection between the terminals 606a-606d of the embedded socket 600 and the recognition electrodes 550 and 570 of the test strips 540 and 560.

FIG. 7 shows another exemplary socket of the device according to the invention. As shown in FIG. 7, the socket 700 has only the terminals 702a-702d connected with the recognition electrode of a test strip, and does not have a reference electrode and a working electrode differently from the socket 600 shown in FIG. 6. Therefore, the socket 700 is used only for a photometric test strip on which a recognition electrode is formed. For example, when the electrochemical test strip 500 shown in FIG. 5a is inserted into the socket 700, the recognition electrode 510 is electrically connected with the terminal 702a, and the device identifies that the inserted test strip is an electrochemical test strip for measuring glucose and display this fact on the LCD 314 (shown in FIG. 3a ). Contrary to this, when the photometric test strip 540 shown in FIG. 5c is inserted into the socket 700, the recognition electrode 550 is electrically connected with the terminal 702c and the device identifies that the inserted test strip is a photometric test strip for measuring glucose. And then, the device reads the appropriate analysis algorithm from the memory and analyzes the reaction result of the test strip 540. In other words, the device can identify the insertion of a test strip, the analysis method, and the target material of the inserted test strip through the socket 700.

FIG. 8 shows the third exemplary socket of the device according to the invention. As shown in FIG. 8, the socket 800 has only the micro-switch 802 and does not have a terminal for a recognition electrode. Therefore, the socket 800 is used only for the conventional photometric test strip on which no recognition electrode is formed. Since the socket 800 does not have a terminal for a recognition electrode differently from the socket 600 and 700 shown respectively in FIG. 6 and FIG. 7, the device can identify merely whether a test strip is inserted or not through the socket 800. When the micro-ball 804 is pushed up by the insertion of a test strip, the micro-switch 802 generates a signal that indicates the insertion of a test strip. Since the manufacturing of an electrochemical requires patterning of electrodes on an insulated substrate in order to make a reference electrode and a working electrode, a recognition electrode can be patterned at the same time with the reference electrode and the working electrode. However, the conventional photometric test strip does not require such a patterning process, so the patterning process for forming a recognition electrode must be added in manufacturing a photometric test strip. The socket 800 shown in FIG. 8 is useful to the device for analyzing only one analyte with a photometric method, because the socket 800 can inform the measuring device only whether a test strip is inserted or not, without any information regarding target material.

FIG. 9 is the fourth exemplary socket of the device according to the invention. As shown in FIG. 9, the socket 900 has the reference electrode 902, the working electrode 904, the recognition electrodes 906a-906d, and the micro-switch 908. Therefore, the socket 900 can be used for not only the electrochemical/photometric test strip with a recognition electrode, but also the photometric test strip without a recognition electrode. Through the micro-switch 908, the device can identify the insertion of a photometric test strip without a recognition electrode. And in case of the photometric test strip with a recognition electrode, through the recognition electrode, the device can be informed of the target material, the analysis method, the insertion of a test strip, and so forth.

FIG. 10 shows a flow chart for explaining the control method of the device according to this invention. When a user presses a start button, the device starts the initial state (step 1002), displays an icon indicating whether a test strip is inserted or not on LCD, and then checks the insertion of a test strip (step 1004). Then, when a test strip is inserted, the device rings a buzzer and identifies the position or the existence of the recognition electrode patterned on the inserted test strip so as to discriminate the analysis method (steps 1008 and 1010). If the inserted test strip is electrochemical one, the device drives the unit for causing a redox reaction in the test strip (step 1012). On the other hand, if the inserted test strip is photometric one, the device drives the unit for sensing the color change of the reaction region (step 1014). If the inserted test strip is neither photometric one nor electrochemical one, the device shows an error message on LCD (step 1015) and waits for the insertion of a new test strip (steps 1032 and 1034).

Next, the device identifies the target material to be analyzed from the position of the recognition electrode (steps 1016, 1018, 1020 and 1022), and then executes the corresponding analysis routine (steps 1024, 1026, 1028 and 1030). For example, if the inserted test strip is electrochemical one for measuring glucose, the device executes a processing routine for an electrochemical glucose strip (step 1024). When a test strip is removed after the execution of a processing routine and a predetermined time lapses without the insertion of a new test strip, the device is automatically turned off. If a new test strip is inserted, the device rings a buzzer and re-executes the step of identifying the type of a test strip (steps 1032, 1034, 1006, 1008 and 1010). In the step 1004, the device checks whether a memory button is pressed as well as whether a test strip is inserted. If the memory button is pressed, it may further fulfill the step of reading the value stored in the memory unit of the device, such as EEPROM (electrically erasable and programmable read only memory). When the test strip is removed and a prescribed time lapses without insertion of a new test strip, the device rings buzzer and is automatically turned off (steps 1032, 1034, 1036, 1038, 1040 and 1042). In case that a test strip is removed from the device in the middle of measuring, the device re-executes the processing routine from step 1004.

The processing routine for an electrochemical glucose strip at step 1024 is executed concretely through the following steps. Firstly, the device displays the icon representing the target material is glucose on LCD, and then blinks the icon that instructs the user to apply a sample, such as blood, into the reaction region of a test strip. When a sample is applied into a test strip, a low voltage is impressed between the reference electrode and the working electrode during a predetermined time (for example, 8 seconds) so as to produce a reaction between the sample and the reagent. After 8 seconds passed, the voltage of about 0.3V is impressed between the reference electrode and the working electrode on the inserted test strip during a predetermined time (for example, 3 seconds). Subsequently, the device measures the electric current flowing in the working electrode and converts the value of the measured current into glucose level by using the relation table stored in the memory of the device. Then, the device stores the glucose level in EEPROM and indicates it on LCD. The processing routine for an electrochemical cholesterol strip at step 1026 is the same as the processing routine at step 1024, except that the relation between the measured current and the cholesterol level is different from that of the processing routine at step 1024.

The processing routine for a photometric glucose strip at step 1028 is executed as follows. Firstly, the device displays the icon representing the target material is glucose on LCD, and blinks the icon that instructs the user to apply a sample into the reaction region on the inserted test strip. A sample is applied to the test strip with the test strip off the device or with the test strip inserted into the device. The device checks whether a sample is applied by using a light-emitting element such as LED and a light-detecting element such as a photo detector. When a sample is applied, the device waits till the intensity of light reflected from the reaction region is stabilized. When the reaction is stabilized, the device converts the intensity of the reflected light into the glucose level, stores this level in EEPROM and displays it on LCD. The processing routine for a photometric cholesterol strip at step 1030 is the same as the processing routine at step 1028, except that the relationship between the intensity of the reflected light and the cholesterol level is different from that of the processing routine at step 1028.

All patents and other publications specifically identified in this specification are indicative of the level of sill of those of ordinary sill in the art to which this invention pertains and are herein individually incorporated by reference to the same extent as would occur if each reference were specifically and individually incorporated by reference.

The invention now being fully described, it will be apparent to one of ordinary skill in the art that many modifications and changes can be made thereto without departing from the spirit or scope of the invention as defined in the following claims.

### INDUSTRIAL APPLICABILITY

According to the invention, it is possible to measure various biochemical components in one device regardless of the type of a test strip, namely, electrochemical or photometric one. Therefore, the device can perform the analysis of high accuracy for some materials to which the electrochemical method can be applicable, and perform analysis for the other various materials by using the photometric method. In addition, the device can automatically identify the type of a test strip (namely, the analysis method) and the target material, without an additional operation of a button, by using the recognition electrode of the test strip and the terminals for the recognition electrode in the socket of the device. The test strip according to the invention can be manufactured more inexpensively than the conventional one because the recognition electrode is merely an electrode formed on the surface of a test strip differently from the code chip.

1. A biochemical components analyzing device which is used together with a test strip, comprising:
a first socket into which a photometric test strip is inserted;
a second socket into which an electrochemical test strip is inserted;
means for analyzing the biochemical components with a photometric method when the biochemical components is applied in the photometric test strip;
means for analyzing the biochemical components with an electrochemical method when the biochemical components are applied in the electrochemical test strip;
means for displaying analysis results from the photometric analyzing means and the electrochemical analyzing means; and
a controller for driving the photometric analyzing means when the photometric test strip is inserted into the first socket, and driving the electrochemical analyzing means when the electrochemical test strip is inserted into the second socket.

2. The biochemical components analyzing device as set forth in claim 1, wherein:
the photometric test strip has a recognition electrode indicating information about target material and analysis method by the position formed on the photometric test strip; and
the first socket includes plural terminals one of which is electrically connected with the recognition electrode, and determines analysis method and target material of the photometric test strip inserted into the first socket according to the position of the terminal that is electrically connected with the recognition electrode.

3. The biochemical components analyzing device as set forth in claim 2, wherein:
the first socket determines whether the photometric test strip is inserted, according to electrical connection between the terminals for the recognition electrode of the first socket and the recognition electrode of the photometric test strip.

4. The biochemical components analyzing device as set forth in claim 1, wherein:
the first socket has a built-in switch that determines whether the photometric test strip is inserted.

5. The biochemical components analyzing device as set forth in claim 1, wherein:
the electrochemical test strip has a reference electrode, a working electrode, and a recognition electrode indicating information about target material and analysis method by the position formed on the electrochemical test strip; and
the second socket includes
a first terminal electrically connected with the working electrode,
a second terminal electrically connected with the reference electrode, and
a plurality of third terminal one of which is electrically connected with the recognition electrode, and determines analysis method and target material of the electrochemical test strip inserted into the second socket according to the position of the third terminal which is electrically connected with the recognition electrode.

6. A biochemical components analyzing device which is used together with a test strip, comprising:
a socket into which a photometric test strip or an electrochemical test strip is inserted selectively;
means for analyzing the biochemical components with a photometric method when the biochemical components are applied in the photometric test strip;
means for analyzing the biochemical components with an electrochemical method when the biochemical components are applied in the electrochemical test strip;
means for displaying analysis results from the photometric analyzing means and the electrochemical analyzing means; and
a controller for driving the photometric analyzing means when the photometric test strip is inserted into the socket, and driving the electrochemical analyzing means when the electrochemical test strip is inserted into the socket.

7. The biochemical components analyzing device as set forth in claim 6, wherein:
the photometric test strip has a first recognition electrode indicating information about target material and analysis method by the position formed on the photometric test strip,
the electrochemical test strip has a reference electrode, a working electrode, and a second recognition electrode indicating information about target material and analysis method by the position formed on the electrochemical test strip, and
the socket includes
a first terminal electrically connected with the working electrode;
a second terminal electrically connected with the reference electrode; and
a plurality of third terminal one of which is electrically connected with the first recognition electrode or the second recognition electrode, and determines analysis method and target material of the test strip inserted into the socket according to the position of the third terminal which is electrically connected with the first recognition electrode or the second recognition electrode.

8. The biochemical components analyzing device as set forth in claim 7, wherein:
the socket determines whether the test strip is inserted according to electrical connection between the third terminal of the socket and the recognition electrode of the test strip inserted into the socket.

9. The biochemical components analyzing device as set forth in claim 7, wherein:
the socket has a built-in switch that determines whether the photometric test strip is inserted.

10. The biochemical components analyzing device as set forth in claim 6, wherein:
the electrochemical test strip has a reference electrode, a working electrode, and a recognition electrode indicating information about target material and analysis method by the position formed on the electrochemical test strip; and
the socket includes
a first terminal electrically connected with the working electrode,
a second terminal electrically connected with the reference electrode,
a plurality of third terminal one of which is electrically connected with the recognition electrode, and determines analysis method and target material of the test strip inserted into the socket according to the position of the third terminal which is electrically connected with the recognition electrode, and
a built-in switch that determines whether the test strip is inserted.
